(19) United States
(12) Patent Application Publication
(10) Pub. No.: US 2014/0193463 A1
(43) Pub. Date: Jul. 10, 2014

(54) PEPTIDE FOR INHIBITING DIPEPTIDYL-PEPTIDASE IV

(71) Applicant: CHINA MEDICAL UNIVERSITY, Taichung City (TW)

(72) Inventors: Kuo-Chiang HSU, Taichung City (TW); Kit-Pan HO, MACAU (CN); Shih-Li HUANG, Chiayi City (TW); Chia-Ling JAO, Kaohsiung City (TW)

(73) Assignee: CHINA MEDICAL UNIVERSITY, Taichung City (TW)

(21) Appl. No.: 13/733,903

(22) Filed: Jan. 4, 2013

(51) Int. Cl. C07K 5/103; C12N 9/99

(52) U.S. Cl. CPC C07K 5/1008; C12N 9/99
USPC 424/400; 435/184; 530/330; 514/7.2

(57) ABSTRACT

A method for inhibiting dipeptidyl-peptidase IV is provided and includes administering a peptide to the dipeptidyl-peptidase IV, in which the peptide is isolated from a gelatin hydrolysate produced by enzymatic digestion with alcalase (ALA), bromelain (BRO), and flavourzyme (FLA).

### SEQUENCE LISTING

The sequence listing submitted via EFS, in compliance with 37 CFR §1.52(e)(5), is incorporated herein by reference. The sequence listing text file submitted via EFS contains the file “TWT02438US-SequenceListing”, created on Nov. 7, 2012, which is 730 bytes in size.

### BACKGROUND

1. Field of Invention

The present disclosure relates to a peptide having peptidase inhibitory activities. More particularly, the present disclosure relates to a peptide having dipeptidyl-peptidase IV inhibitory activities.

2. Description of Related Art

During a meal, two incretin hormones, glucose-dependent insulinotropic polypeptide (GIP) and glucagon-like peptide-1 (GLP-1), are released from the small intestine into the vasculature and augment glucose-induced insulin secretion from the islet β-cells. It has been reported that approximately 50-60% of the total insulin secreted during a meal results from the incretin response, mainly the effects of GIP and GLP-1. However, GIP and GLP-1 had extremely short half-lives of about 1-2 min following secretion due to the rapid degradation and inactivation by the enzyme dipeptidyl-peptidase IV (DPP-IV), resulting in loss of their insulinotropic activities. It has been reported that most of the degraded GLP-1 is attributed to the action of DPP-IV. Therefore, the use of DPP-IV inhibitors as a new therapeutic approach for the management of type 2 diabetes was also developed. Some studies on the administration of DPP-IV inhibitors in animal and clinical experiments have shown increased half-life of total circulating GLP-1, decreased plasma glucose, and improved impaired glucose tolerance, Dipeptidyl-peptidase IV (DPP-IV) is a postproline-cleaving enzyme with the specificity for removing X-proline or X-alanine dipeptides from the N-terminus of polypeptides. The cleavage of N-terminal peptides with Pro in the second position is a rate-limiting step in the degradation of peptides. There are several chemical compounds used in vitro and in animal models to inhibit DPP-IV activity, such as valine-pyrrolidide, NVP-DPP728, and Lys [Z(NO₂)]-thiazolidide and Lys[Z(NO₂)]-pyrrolidide. However, such chemical compounds, which often have to be administered by injection, may result in side effects as chemical drugs. Diprotins A and B, isolated from culture filtrates of Bacillus cereus BMF673-RF1, were found to exhibit the inhibitory activity on DPP-IV with IC₅₀ values of 1.1 and 5.5 μg/mL, respectively. They were elucidated to be Ile-Pro-Ile and Val-Pro-Leu. There were also two peptides, Ile-Pro-Ala and Val-Ala-Gly-Thr-Trp-Tyr, prepared from β-lactoglobulin hydrolyzed by proteinase K and trypsin, which showed IC₅₀ values of 49 and 174 μM against DPP-IV, respectively.

Patents WO 2006/068480 and WO 2009/128713 have demonstrated that the peptides derived from casein and lysozyme hydrolysates display DPP-IV-inhibiting activity, and the peptides how in particular the presence of at least one proline within the sequence and mostly in the second N-terminal residue.

It is known that the dominant amino acid in gelatin is glycine, whereas the imino acids (proline and hydroxyproline) come second in abundance. The amino acid composition is characterized by a repeating sequence of Gly-X—Y triplets, where X is mostly proline and Y is mostly hydroxyproline. Furthermore, some studies have reported that the DPP-IV inhibitory peptides consisted of at least one proline and mostly as the penultimate N-terminal residue.

### SUMMARY

An aspect of the present disclosure is to provide peptides having DPP-IV-inhibitory activities. These peptides are mainly artificially synthesized or isolated from Atlantic salmon skin gelatin. This is expected to give insight into the possible utilization of Atlantic salmon skin as a potential source of DPP-IV inhibitors that can be used in the prevention and/or the treatment of type 2 diabetes.

According to a first embodiment of the present disclosure, a method for inhibiting dipeptidyl-peptidase IV includes administering a peptide to the dipeptidyl-peptidase IV, in which the peptide is isolated from a gelatin hydrolysate produced by enzymatic digestion with alcalase (ALA), bromelain (BRO), or flavourzyme (FLA).

The aforementioned peptide contains four amino acid residues and has an amino acid sequence of Gly-Pro-X₃-X₄ (SEQ ID NO: 1), in which X₃ is alanine or glycine, and X₄ is glutamate or alanine. Further, the aforementioned gelatin is an Atlantic salmon (Salmo salar) skin gelatin.

According to a second embodiment of the present disclosure, a peptide includes four amino acid residues having an amino acid sequence of Gly-Pro-X₃-X₄ (SEQ ID NO: 1), in which X₃ alanine or glycine, and X₄ is glutamate or alanine.

According to a third embodiment of the present disclosure, a method for inhibiting dipeptidyl-peptidase IV includes administering a synthesized peptide to the dipeptidyl-peptidase IV, in which the synthesized peptide contains four amino acid residues and has an amino acid sequence of Gly-Pro-X₃-X₄ (SEQ ID NO: 1), in which X₃ is alanine or glycine, and X₄ is glutamate or alanine.

According to a fourth embodiment of the present disclosure, a method for treating type 2 diabetes includes administering an effective amount of a peptide to an animal, in which the peptide contains four amino acid residues and has an amino acid sequence of Gly-Pro-X₃-X₄ (SEQ ID NO: 1), in which X₃ is alanine or glycine, and X₄ is glutamate or alanine.

According to a fifth embodiment of the present disclosure, a pharmaceutical composition includes a peptide including four amino acid residues having an amino acid sequence of Gly-Pro-X₃-X₄ (SEQ ID NO: 1), in which X₃ is alanine or glycine, and X₄ is glutamate or alanine.

### BRIEF DESCRIPTION OF THE DRAWINGS

The invention can be more fully understood by reading the following detailed description of the embodiment, with reference made to the accompanying drawings as follows:

FIG. 1A illustrates DHs (degree of hydrolysis) of Atlantic salmon skin gelatin hydrolyzed separately with ALA, BRO, and FLA at various E/S ratios for 4 h according to an example of the present disclosure;

FIG. 18 illustrates DPP-IV inhibitory activities of Atlantic salmon skin gelatin hydrolyzed separately with ALA, BRO, and FLA at various E/S ratios for 4 h according to an example of the present disclosure;

FIG. 2A illustrates DPP-IV inhibitory activities of 6% FLA hydrolysate fractions separated by ultrafiltration at the concentration of 2 mg solid/mL according to an example of the present disclosure;

FIG. 2B illustrates a DPP-IV inhibition rate of a <1 kDa ultrafiltration fraction of 6% FLA hydrolysate at various concentrations according to an example of the present disclosure;

FIG. 3A is an elution profile of peptide fractions separated from a <1 kDa ultrafiltration fraction of 6% FLA hydrolysate according to an example of the present disclosure;

FIG. 3B illustrates DPP-IV inhibitory activities of peptide fractions separated from a <1 kDa ultrafiltration fraction of 6% FLA hydrolysate according to an example of the present disclosure;

FIG. 3C illustrates a DPP-IV inhibitory activity of a fraction F-1 separated from a <1 kDa ultrafiltration fraction of 6% FLA, hydrolysate according to an example of the present disclosure;

FIG. 4A illustrates a DPP-IV inhibitory activity of a synthetic peptide, Gly-Pro-Ala-Glu, according to an example of the present disclosure;

FIG. 4B illustrates a DPP-IV inhibitory activity of another synthetic peptide, Gly-Pro-Gly-Ala, according to an example of the present disclosure; and

FIG. 4C illustrates a DPP-IV inhibitory activity of diprotin A according to an example of the present disclosure.

### DETAILED DESCRIPTION

In the following detailed description, for purposes of explanation, numerous specific details are set forth in order to provide a thorough understanding of the disclosed embodiments. It will be apparent, however, that one or more embodiments may be practiced without these specific details.

A peptide is provided and includes four amino acid residues. The peptide has an amino acid sequence of Gly-Pro-X₃-X₄ (SEQ ID NO: 1), in which X₃ is alanine or glycine, and X₄ is glutamate or alanine.

A method is provided for inhibiting dipeptidyl-peptidase IV (DPP-IV), in which the method includes administering a peptide to the dipeptidyl-peptidase IV, whether inn vivo or in vitro. The aforementioned peptide is isolated from an Atlantic salmon (Salmo salar) skin gelatin hydrolysate produced by enzymatic digestion with alcalase (ALA), bromelain (BRO), or flavourzyme (FLA). The peptide includes four amino acid residues having an amino acid sequence of Gly-Pro-X₃-X₄ (SEQ ID NO: 1), in which X₃ is alanine or glycine, and X₄ is glutamate or alanine.

The peptide is isolated from a fraction of the Atlantic salmon skin gelatin hydrolysate fractionated by ultrafiltration with 1 kDa and 2.5 kDa cutoff membranes, in which the fraction includes the fractionated Atlantic salmon skin gelatin hydrolysate permeating through the 1 kDa cutoff membrane.

Another method for inhibiting dipeptidyl-peptidase IV is provided, and this method includes administering a synthesized peptide to the dipeptidyl-peptidase IV, in which the synthesized peptide includes four amino acid residues and has an amino acid sequence of Gly-Pro-X₃-X₄ (SEQ ID NO: 1) in which X₃ is alanine or glycine, and X₄ is glutamate or alanine.

A method for treating type 2 diabetes is also provided and includes administering an effective amount of a peptide to an animal, in which the peptide includes four amino acid residues and has an amino acid sequence of Gly-Pro-X₃-X₄ (SEQ ID NO; 1), in which X₃ is alanine or glycine, and X₄ is glutamate or alanine. The aforementioned animal can be a mammal. More particularly, the mammal can be a human being.

In addition, a pharmaceutical composition is provided and includes a peptide having four amino acid residues having an amino acid sequence of Gly-Pro-X₃-X₄ (SEQ ID NO: 1), in which X₃ is alanine or glycine, and X₄ is glutamate or alanine. The pharmaceutical composition may further comprise a pharmaceutically suitable carrier, a pharmaceutically acceptable salt, and/or a pharmaceutically acceptable excipient. The pharmaceutical composition can be in the form of a tablet, capsule, pill, syrup, suspension, solution, powder, granule, emulsion, microsphere, injectable solution, solid lipid nanoparticles, a feed, a drink, a nutrient supplement, a food, or a healthy food.

### EXAMPLES

The following examples are described for those skilled in the art to further understand the present disclosure and should not be limited to the present disclosure.

### Materials and Reagents

Atlantic salmon (Salmo salar) fish skins, processing byproducts recovered from fresh skin-off fillets, were supplied by Albion Fisheries Ltd. (Vancouver, BC, Canada). The fish skins were transferred on ice, vacuum packed, and stored at −25° C. until use. Three food-grade proteolytic enzymes were donated by Neova Technologies Inc. (Abbotsford, BC, Canada). Alcalase 2.4 L FG (from Bacillus licheniformis, 2.4 AU/g) and Flavourzyme 1000 L (from Aspergillus oryzae, 1000 LAPU/g) were products from Novo-zymes North America Inc. (Salem, N.C., Canada), whereas bromelain (from pineapple stem, 2000 GDU/g) was manufactured by Ultra Bio-Logics Inc. (Montreal, QC, Canada), Dipeptidyl-peptidase IV (D7052, from porcine kidney), Gly-Prop-nitroanilide hydrochloride, trichloroacetic acid (TCA), L-leucine, and diprotin A were purchased from Sigma-Aldrich (St. Louis, Mo.). Trinitrobenzenesulfonic acid (TNBS) was obtained from Fluka Biochemika Oakville ON, Canada). Other chemicals and reagents used were of analytical grade and commercially available,

### Statistical Analysis

Each data point represents the mean of three samples subjected to analysis of variance (ANOVA) followed by Tukey's studentized range test, and the significance level of P<0.05 was employed.

### Extraction of Gelatin

The thawed skins were gently washed with running tap water, drained, and cut into pieces (of about 5×10 cm in size). The fish skins were soaked in 0.2 M NaOH (1:10; w/v) and stirred in a cold room at 4° C. for 30 min. This procedure was repeated three times to remove noncollagenous proteins and pigments. The skins were washed with running tap water until the pH was neutral. Afterwards, the skins were soaked in 0.05 M acetic acid (1:10; w/v), stirred at room temperature for 3 h, and then washed by running tap water until the pH was neutral. Almost all of the scales could be removed. The gelatin of the swollen skins was extracted in distilled, deionized water (ddH₂O; 1:2; w/v) at 70° C. for 3 h. The oil and aqueous layers of the gelatin extract were separated by separatory funnels, and the gelatin extract was filtered through two layers of cheesecloth, lyophilized, and stored in a desiccator until use.

### Amino Acid Analysis

The gelatin extract was hydrolyzed under vacuum in 6 M HCl (1:1; v/v) at 110° C. for 24 h in the presence of 1% phenol (v/v), and the hydrolysates of the gelatin extract were analyzed using an amino acid analyzer (Hitachi L-8900, Hitachi Ltd., Katsuda, Japan). Moreover, the content of tryptophan was determined by a colorimetric method at 550 nm after alkaline hydrolysis of the gelatin extract at 105° C. for 24 h with 4 M NaOH.

Amino Acid Composition of Atlantic Salmon Skin Gelatin

The amino acid composition of Atlantic salmon skin gelatin is presented in Table 1. The glycine content of salmon skin gelatin was 223.63 mg/g sample, slightly higher than that of Nile tilapia skin gelatin (211.8 mg/g protein) and similar to that of porcine skin gelatin (224.5 mg/g protein). The alanine content (7.06 mol/100 mol amino acids) of salmon skin gelatin in the present example was lower than those (9.6-12.3 mol/100 mol amino acids) of skin gelatins from other fish species, such as cod, Alaska pollock, hake, and tilapia. Salmon skin gelatin contained a high content of imino acids (175.02 mg/g sample), including proline (86.78 mg/g sample) and hydroxyproline (88.24 mg/g sample), which showed slightly lower contents (186.29 and 187.42 mg/g sample) when compared to bigeye snapper skin gelatins.

TABLE 1
Amino Acid Composition of Gelatin from Atlantic Salmon Skins

| | content (n = 3) | |
|---|---|---|
| amino acid | mg/g sample | mol/100 mol amino acids |
| Alanine | 50.24 | 7.06 |
| Arginine | 83.51 | 6 |
| aspartic acid | 78.15 | 7.35 |
| Cysteine | 2.32 | 0.12 |
| glutamic acid | 89.84 | 7.65 |
| Glycine | 223.63 | 37.31 |
| Histidine | 3.18 | 0.26 |
| hydroxyproline (Hyp) | 88.24 | 8.43 |
| Isoleucine | 9.61 | 0.92 |
| Leucine | 21.83 | 2.08 |
| Lysine | 33.18 | 2.84 |
| Methionine | 16.26 | 1.36 |
| Phenylalanine | 18.24 | 1.38 |
| proline (Pro) | 86.78 | 9.44 |
| Serine | 27.21 | 3.24 |
| Threonine | 26.08 | 2.74 |
| Tryptophan | 1.09 | 0.07 |
| Tyrosine | 6.54 | 0.45 |
| Valine | 11.95 | 1.28 |
| imino aicds (Hyp + Pro) | 175.02 | 17.87 |

### Enzymatic Hydrolysis

One gram of lyophilized gelatin extract with 50 mL of ddH₂O added was incubated at 50° C. for 10 min prior to the enzymatic hydrolysis. Enzymes in liquid form were weighed as 10, 20, 30, 60 mg and mixed with 1 mL of ddH₂O. The hydrolysis reaction was started by the addition of enzymes at various enzyme/substrate ratios (E/S: 1, 2, 3, and 6%). The reactions with alcalase (ALA), bromelain (BRO), and Flavourzyme (FLA) were conducted at pH 8.0, 7.0, and 7.0, respectively, and 50° C. for 4 h. After hydrolysis, the hydrolysates therefrom were heated in boiling water for 10 min to inactivate enzyme activities and then cooled in cold water at room temperature for 20 min. The hydrolysates were adjusted to pH 7.0 with 1 M NaOH and centrifuged (DuPont Small Centrifuge RC 5B, Mandel Scientific Co. Ltd., Guelph, ON, Canada) at 12000 g at room temperature for 15 min. The supernatant was lyophilized and stored at −25° C.

### Measurement of Degree of Hydrolysis (DH)

Immediately prior to termination of the aforementioned hydrolysis, a 4 mL aliquot of the hydrolysate was mixed with an equal volume of 24% TCA solution and centrifuged at 12200 g for 5 min. The supernatant (0.2 mL) was added to 2.0 mL of 0.05 M sodium tetraborate buffer (pH 9.2) and 1 mL of 4.0 mM TNBS, and was incubated at room temperature for 30 min in dark. Then, 1.0 mL of 2.0 M NaH₂PO₄ containing 18 mM Na₂SO₃ was added to the mixture, and the absorbance was measured at 420 nm using a spectrophotometer (Cary 50 Bio UV-vis spectrophotometer, Varian, Inc., Santa Clara, Calif.). The degree of hydrolysis (DH) was calculated as % DH=(h/hₜₒₜₐₗ)×100, where DH=percent ratio of the number of peptide bonds broken (h) to the total number bonds per unit weight (hₜₒₜₐₗ) and hₜₒₜₐₗ=11.1 mequiv/g of gelatin. L-Leucine was used for drawing a standard curve.

### Determination of DPP-IV-Inhibitory Activity

DPP-IV activity determination was performed in 96-well microplates. In order to measure an increment in absorbance at 405 nm, Gly-Pro-p-nitroanilide is used as a DPP-IV substrate. The lyophilized hydrolysates were dissolved in 100 mM Tris buffer (pH 8.0) to the concentration of 10 mg/mL and then serially diluted. The hydrolysates (25 μL) were added with 25 μL of 1.59 mM Gly-Pro-p-nitroanilide (in 100 mM Tris buffer, pH 8.0). The mixture was incubated at 37° C. for 10 min, followed by an addition of 50 μL of DPP-IV (diluted with the same Tris buffer to 0.01 unit/mL). The reaction mixture was incubated at 37° C. for 60 min, and the reaction was suspended by adding 100 μL of 1 M sodium acetate buffer (pH 4.0). The absorbance of the resulting solution was measured at 405 nm with a microplate reader (iEMS reader MF; Labsystems, Helsinki, Finland). IC₅₀ (concentration of 50% inhibition) values were determined by assaying appropriately diluted samples and plotting the DPP-IV inhibition rate as a function of the hydrolysate concentration.

### DH and DPP-IV-Inhibitory Activity of Hydrolysates

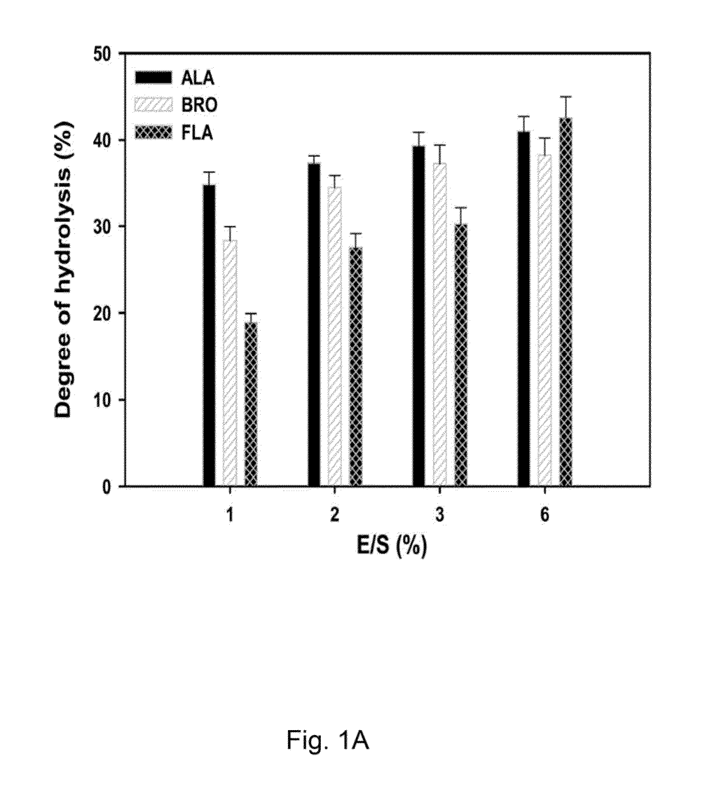

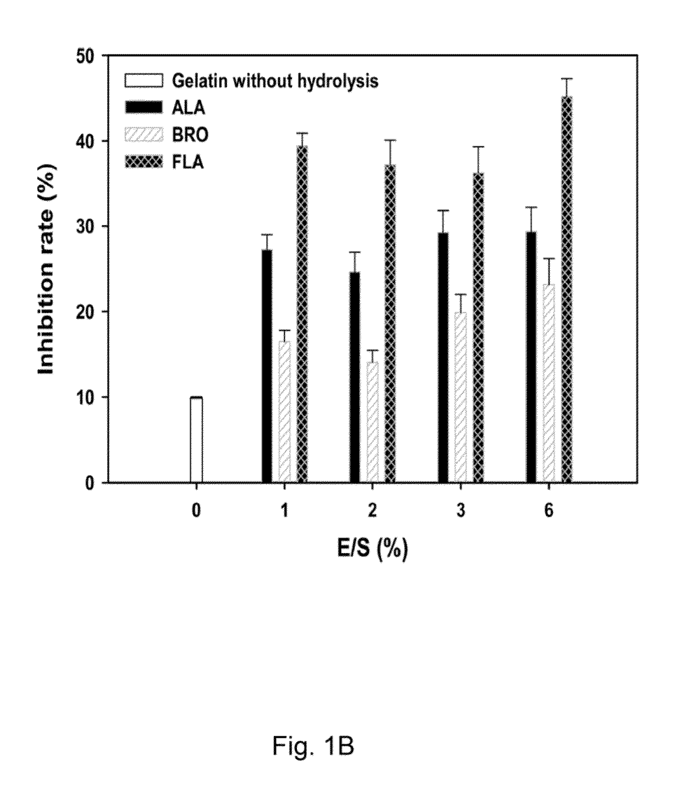

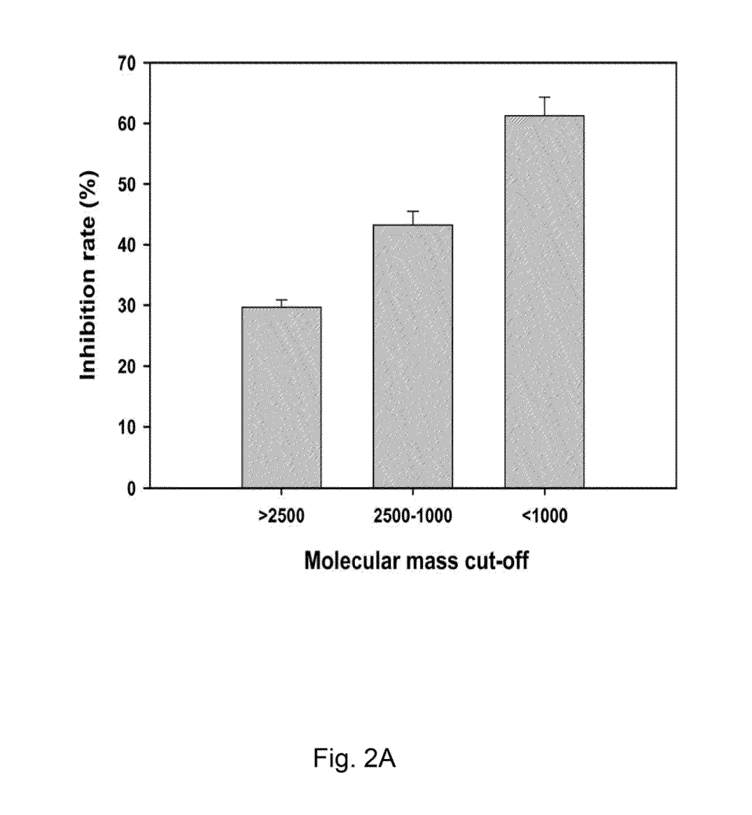

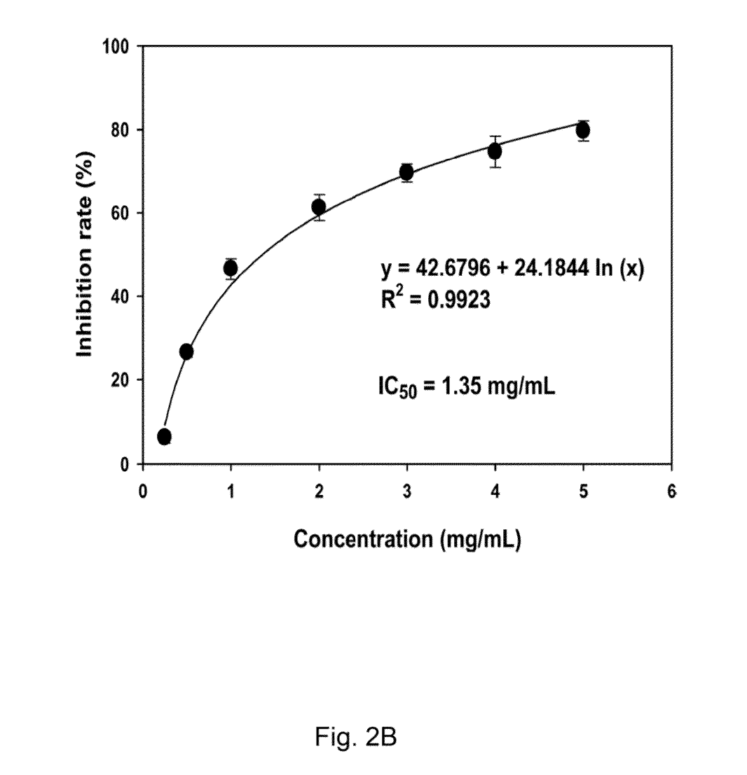

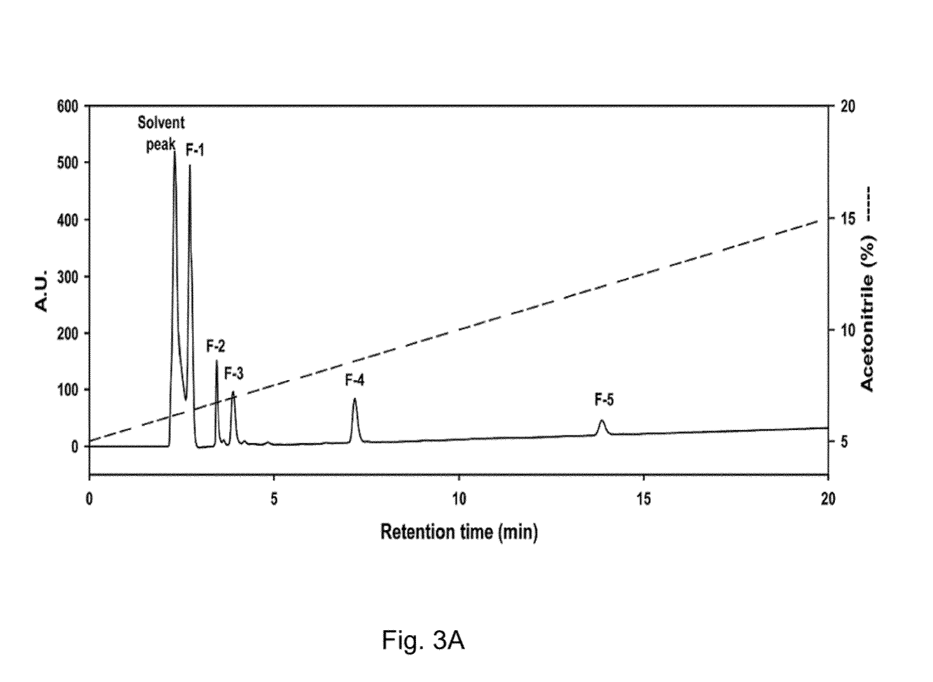

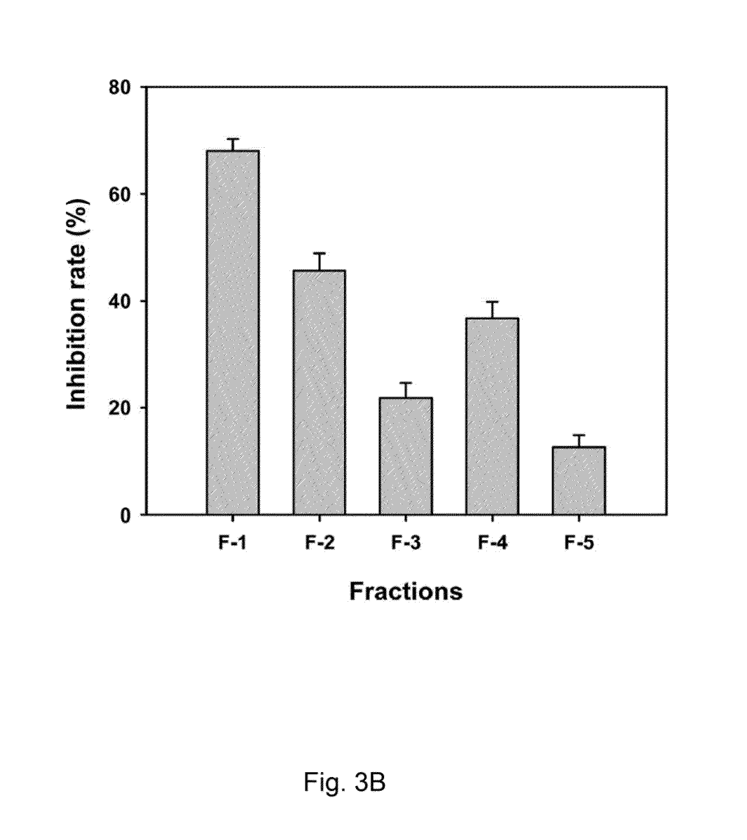

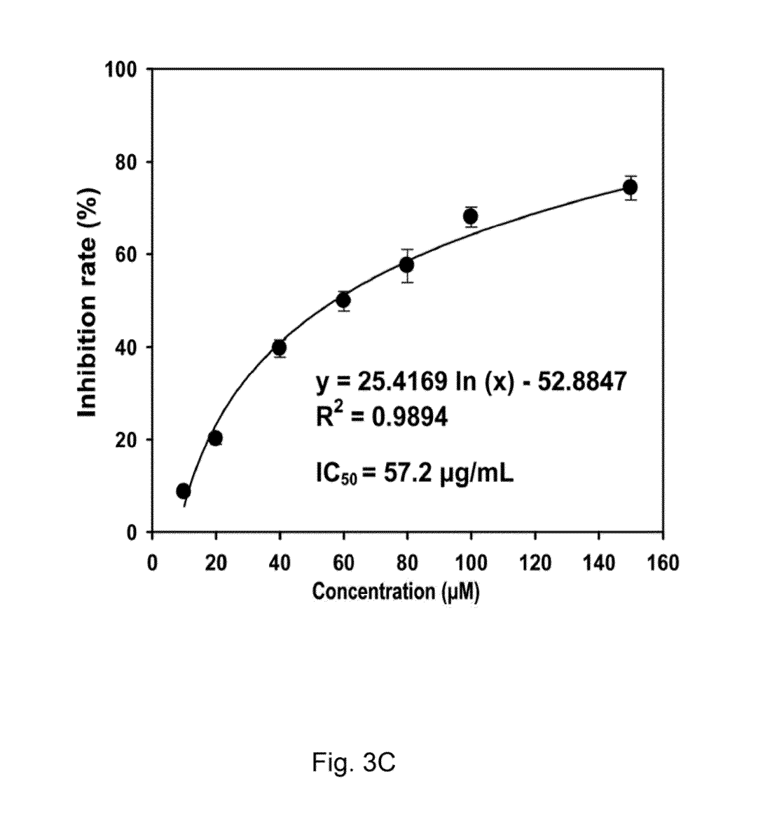

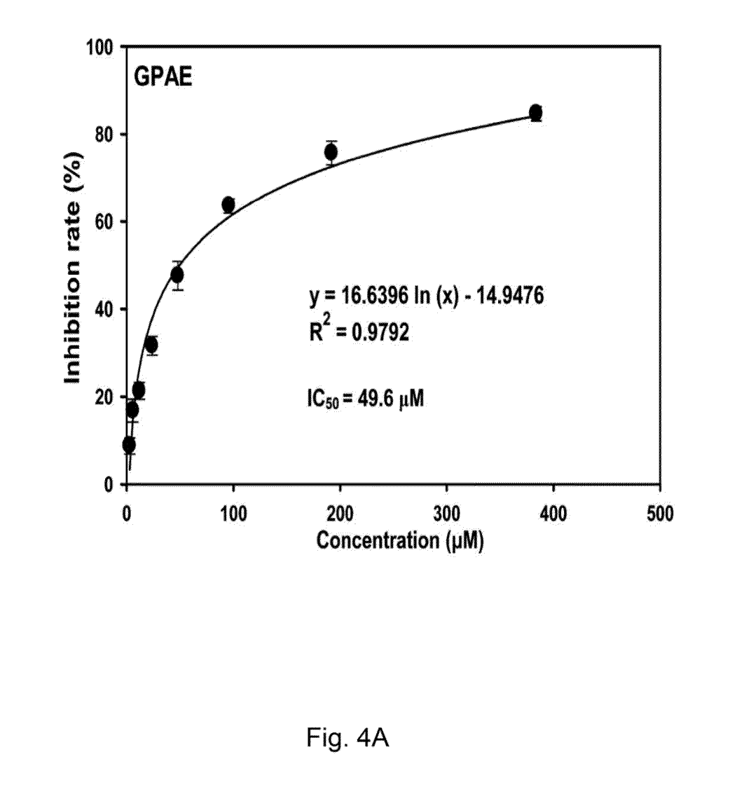

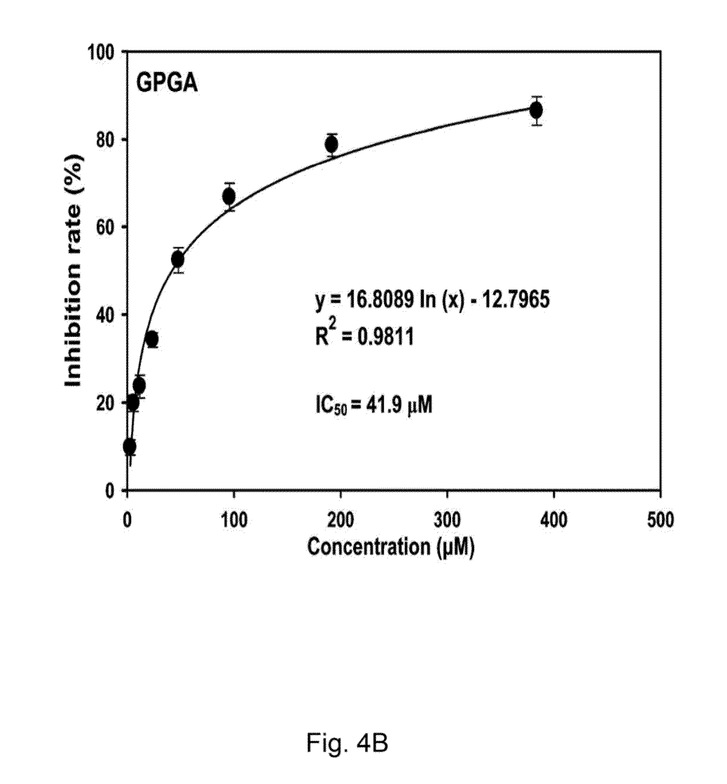

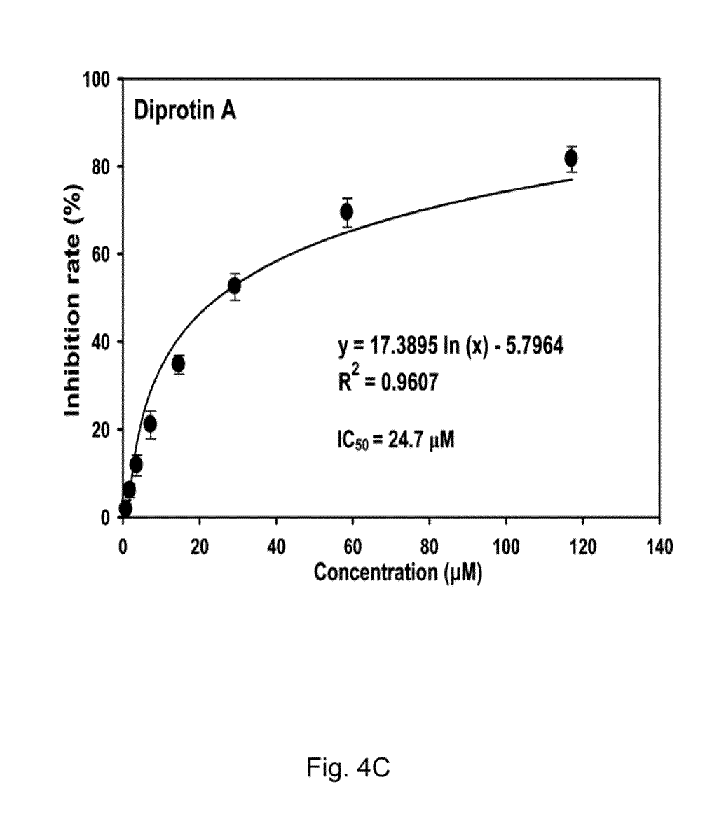

The DHs and DPP-IV-inhibitory activities of Atlantic salmon skin gelatin hydrolyzed separately with ALA (alcalase), BRO (bromelain), and FLA (flavourzyme) at various E/S ratios for 4 h are shown in each of FIG. 1A and FIG. 1B. The three proteases used in the present example were alcalase (a serine protease), bromelain (a cysteine protease), and flavourzyme (an exo- and endopeptidase complex). The result showed that the DHs of the gelatin hydrolysates obtained by hydrolysis with three proteases increased with the increment of E/S ratio (FIG. 1A). The DHs of ALA and BRO hydrolysates with the E/S ratio of 11 were 34.8 and 28.3%, respectively, and those with 6% were 41 and 38.2%, respectively. The DHs of 1%, 2%, and 3% FLA hydrolysates were lower than those of ALA and BRO hydrolysates, and the 6% FLA hydrolysate showed the slightly higher DH of 42.5% than the other two protease hydrolysates. At the concentration of 5 mg solid/mL, the extracted gelatin (without hydrolysis) showed the DPP-IV inhibition rate of about 10%, and the hydrolysates possessed significantly higher DPP-IV-inhibitory activities (P<0.05) than gelatin (see FIG. 1B). The DPP-IV inhibition rates of ALA hydrolysates with all of the various enzyme concentrations were between 24 and 30% with insignificant differences (P>0.05), and that of 6% BRO hydrolysate showed 23.1%, the highest among all BRO samples (P<0.05). The FLA hydrolysates showed the greatest DPP-IV inhibition rates when compared to ALA and BRO hydrolysates with the same E/S ratio, and that with 6% E/S ratio possessed the highest inhibition rate of 45.2% in the present example (P<0.05). Therefore, the FLA hydrolysate with the E/S ratio of 6% was chosen to be used for further purification. Patent WO 2006/068480 has demonstrated that the hydrolysates possessed great DPPIV-inhibitory activities referred to a mixture of peptides derived from hydrolysis of proteins with the percentage of hydrolyzed peptide bonds of most preferably 20-40%. All of the hydrolysates except of those with 1 and 6% E/S ratios of FLA obtained in the present example showed DHs between 27.6 and 40.9%. However, the DPP-IV-inhibitory activities of the two exceptions were higher than those of the other hydrolysates. Thus, it is reasonable to suggest that the DPP-IV-inhibitory activity should be determined by the peptide structures and sequences, and is not dependent upon DHs.

### Ultrafiltration

The DPP-IV-inhibitory peptides of the hydrolysates were fractionated by ultrafiltration (model ABL085, Lian Sheng Tech. Co., Taichung, Taiwan) with spiral wound membranes having molecular mass cutoffs of 2.5 and 1 kDa. The fractions were collected as follows: >2.5 kDa, peptides retained without passing through 2.5 kDa membrane; 1-2.5 kDa, peptides permeating through the 2.5 kDa membrane but not the 1 kDa membrane; <1 kDa, peptides permeating through the 1 kDa membrane. All fractions collected were lyophilized and stored in a desiccator until use.

### DPP-IV-Inhibitory Activity of Hydrolysates Fractionated by Ultrafiltration

FIG. 2A shows the DPP-IV inhibitory activities of 6% FLA hydrolysate fractions separated by ultrafiltration at the concentration of 2 mg solid/mL. The result showed the peptides within the <1 kDa UF fraction had the greatest DPP-IV inhibition rate of 61.2% (P<0.05), whereas those within the 2.5 and 1-2.5 kDa fractions displayed inhibition rates of 29.6 and 43.2%, respectively. The IC₅₀ value of the <1 kDa fraction was determined and found to be 1.35 mg/mL (see FIG. 2B). The result herein is in agreement with former studies using various protein sources that reported the preferable DPP-IV-inhibitory peptides derived from food protein consisted of two to eight amino acid residues, and their molecular weights were presumed to be ranged between 200 and 1000 Da.

### High-Performance Liquid Chromatography (HPLC)

The fractionated hydrolysates by ultrafiltration having DPP-IV-inhibitory activity were further purified using a High Performance Liquid Chromatography system, HPLC (model L-2130 HPLC, Hitachi Ltd., Katsuda, Japan). The lyophilized hydrolysate fraction (100 μg) by gel filtration was dissolved in 1 mL of 0.1% trifluoroacetic acid (TFA), and 90 μL of the mixture was then injected into a column (Zorbax Eclipse Plus C18, 4.6×250 mm, Agilent Techologies Inc., Santa Clara, Calif.) using a linear gradient of acetonitrile (5-15% in 20 min) in 0.1% TFA under a flow rate of 0.7 mL/min. The peptides were detected at 215 nm. Each collected fraction was then lyophilized and stored in a desiccator until use.

### Purification of DPP-IV-inhibitory Peptides by HPLC

FIG. 3A and FIG. 3B illustrate respectively an elution profile and DPP-IV inhibitory activities of the peptide fractions from the <1 kDa UF fraction separated by HPLC. To obtain a sufficient amount of purified peptide, chromatographic separations were performed repeatedly. Five fractions (F-1-F-5) were obtained upon HPLC separation of the <1 kDa UF fraction (see FIG. 3A), and they were lyophilized and then used to determine their DPP-IV-inhibitory activities at the concentration of 100 μg solid/mL. The result shows that fraction F-1 had the highest DPP-IV inhibition rate of 68.0% (P<0.05) (see FIG. 3B), and the IC₅₀ value of fraction F-1 was also determined as 57.3 μg/mL (see FIG. 3C). Therefore, fraction F-1 was chosen to be used for identifying the amino acid sequences of the peptides contained therein.

### Determination of Amino Acid Sequence

An accurate molecular mass and amino acid sequence of the purified peptides was determined using a Q-TOF mass spectrometer (Micromass, Altrincham, U.K.) coupled with an electrospray ionization (ESI) source. The purified peptides were separately infused into the electrospray source after being dissolved in methanol/water (1:1, v/v), and the molecular mass was determined by the doubly charged (M+2H)⁺² state in the mass spectrum. Automated Edman sequencing was performed by standard procedures using a 477-A protein sequencer chromatogram (Applied Biosystems, Foster City, Calif.).

### Amino Acid Sequence of DPP-IV-Inhibitory Peptides

Two peptides were identified in fraction F-1, and their amino acid sequences were Gly-Pro-Ala-Glu (372.4 Da) and Gly-Pro-Gly-Ala (300.4 Da) as shown in Table 2.

TABLE 2
Amino Acid Sequences of Purified DPP-IVInhibitory Peptides Derived
from Atlantic Salmon Skin Gelatin Hydrolyzed with FLA

| sequence | molecular mass |
|---|---|
| Gly-Pro-Ala-Glu | 372.4 |
| Gly-Pro-Gly-Ala | 300.4 |

### Peptide Synthesis

Peptides were prepared by the conventional. Fmoc solid-phase synthesis method with an automatic peptide synthesizer (model CS 136, CS Bio Co., San Carlos, Calif.), and their purity was verified by analytical RP-HPLC-MS/MS.

### DPP-IV-Inhibitory Activity of the Synthetic Peptides

FIG. 4A illustrates a DPP-IV-inhibitory activity of a synthetic peptide, FIG. 4B illustrates a DPP-IV-inhibitory activity of another synthetic peptide, and FIG. 4C illustrates a DPP-IV inhibitory activity of diprotin A, each at various concentrations. The IC₅₀ value was calculated for each of the peptides. Diprotin A is a well-known peptide having the greatest DPP-IV-inhibitory activity, and the IC₅₀ value of diprotin A was 24.7 μM in the present example (see FIG. 4C). The IC₅₀ values of the two synthetic peptides, Gly-Pro-Ala-Glu and Gly-Pro-Gly-Ala, were 49.6 and 41.9 μM, respectively. In previous studies, the IC₅₀ values against DPP-1V of diprotins A and β isolated from culture filtrates of B. cereus BMF673-RF1 were 3.2 and 16.8 μM, respectively. Moreover, Ile-Pro-Ala and Val-Ala-Gly-Thr-Trp-Tyr, both prepared from β-lactoglobulin, showed IC50 values of 49 and 174 μM against DPP-IV, respectively. Patent WO 2006/068480 reported that diprotin A showed an 1050 value of about 5 μM against DPP-IV, and five peptides, HPIK, LPLP, LPVP, MPLW, and GPFP, comprised four amino acids with Pro as the penultimate N-terminal residue displayed their IC₅₀ values between 76 and 120 μM.

The results showed that the two peptides, Gly-Pro-Ala-Glu and Gly-Pro-Gly-Ala, of the present example showed a lower DPP-IV inhibitory activity lower only than diprotin A and B. However, inhibitory effects the two peptides of the present disclosure were greater than that of other peptides comprising four or more amino acid residues.

In conclusion, two peptides were isolated, Gly-Pro-Ala-Glu and Gly-Pro-Gly-Ala, from Atlantic salmon skin gelatin hydrolysates having inhibitory activity against DPP-IV. These two peptides may be useful for the therapy or prevention of type 2 diabetes.

Although the present disclosure has been described in considerable detail with reference to certain embodiments thereof, other embodiments are possible. Therefore, the spirit and scope of the appended claims should not be limited to the description of the embodiments contained herein.

It will be apparent to those skilled in the art that various modifications and variations can be made to the structure of the present disclosure without departing from the scope or spirit of the invention. In view of the foregoing, it is intended that the present disclosure cover modifications and variations of this invention provided they fall within the scope of the following claims.

1. A method for inhibiting dipeptidyl-peptidase IV comprising:
administering an effective amount of a peptide to the dipeptidyl-peptidase IV, wherein the peptide is isolated from a gelatin hydrolysate produced by enzymatic digestion with bromelain (BRO), or flavourzyme (FLA) and comprises four amino acid residues with an amino acid sequence of
Gly-Pro-X₃-X₄ (SEQ ID NO: 1), wherein
X₃ is alanine or glycine; and
X₄ is glutamate or alanine.

2. (canceled)

3. (canceled)

4. The method according to claim 1, wherein the peptide is isolated from a fraction of the gelatin hydrolysate fractionated by ultrafiltration with 1 kDa and 2.5 kDa cutoff membranes.

5. The method according to claim 4, wherein the fraction comprises the fractionated gelatin hydrolysate permeating through the 1 kDa cutoff membrane.

6. The method according to claim 1, wherein the gelatin is a salmon skin gelatin.

7. The method according to claim 6, wherein the salmon is an Atlantic salmon (Salmo salar).

8. The method according to claim 1, wherein the method is administered in vivo or in vitro.

9. (canceled)

10. (canceled)

11. A method for treating type 2 diabetes, comprising administering an effective amount of a peptide to an animal, wherein the peptide isolated from a gelatin hydrolysate produced by enzymatic digestion with bromelain (BRO), or flavourzyme (FLA) and comprises four amino acid residues and has an amino acid sequence of
Gly-Pro-X₃-X₄ (SEQ ID NO: 1), wherein
X₃ is alanine or glycine; and
X₄ glutamate or alanine.

12. The method according to claim 11, wherein the animal is a mammal.

13. The method according to claim 12, wherein the mammal is a human being.

14. A pharmaceutical composition comprising a peptide isolated from a gelatin hydrolysate produced by enzymatic digestion with bromelain (BRO), or flavourzyme (FLA) includes four amino acid residues having an amino acid sequence of
Gly-Pro-X₃-X₄ (SEQ ID NO: wherein
X₃ is alanine or glycine; and
X₄ is glutamate or alanine.

15. The pharmaceutical composition according to claim 14, further comprising a pharmaceutically suitable carrier.

16. The pharmaceutical composition according to claim 14, further comprising a pharmaceutically acceptable salt.

17. The pharmaceutical composition according to claim 14, further comprising a pharmaceutically acceptable excipient.

18. The pharmaceutical composition according to claim 14, wherein the pharmaceutical composition is in the form of a tablet, capsule, pill, syrup, suspension, solution, powder, granule, emulsion, microsphere, injectable solution or solid lipid nanoparticles.

19. The pharmaceutical composition according to claim 14, wherein the pharmaceutical composition is in the form of a feed, a drink, a nutrient supplement, a food, or a healthy food.


SEQUENCE LISTING (1 sequence)

SEQ ID NO: 1 - protein, 4 aa, Salmo salar
  MISC_FEATURE (3)..(3): Xaa=Ala or Gly
GPXX